(19) United States
(12) Patent Application Publication
(10) Pub. No.: US 2025/0235536 A1
(43) Pub. Date: Jul. 24, 2025

(54) COMPOSITION FOR ULTRASOUND ABLATION

(71) Applicant: EXACT THERAPEUTICS AS, OSLO (NO)

(72) Inventors: Svein KVÅLE, Ås (NO); Melina MÜHLENPFORDT, Trondheim (NO); Ola MYHRE, Haslum (NO); Morten Smedsrud WIGEN, Oslo (NO); Andrew John HEALEY, Moss (NO)

(21) Appl. No.: 19/172,502

(22) Filed: Apr. 7, 2025

Related U.S. Application Data
(63) Continuation of application No. PCT/NO2023/060062, filed on Oct. 4, 2023.

(30) Foreign Application Priority Data
Oct. 5, 2022 (NO) 20221061

(51) Int. Cl. A61K 41/00; A61K 9/00; A61N 7/00; A61N 7/02; A61P 35/00

(52) U.S. Cl. CPC A61K 41/0028; A61K 9/0019; A61N 7/02; A61P 35/00; A61N 2007/003; A61N 2007/0039; A61N 2007/0078; A61N 7/022; A61N 2007/025

(57) ABSTRACT

Disclosed herein is a composition for enhanced ultrasound ablation and methods relating to same. The composition may include a microbubble-microdroplet cluster composition, wherein the microbubble-microdroplet cluster composition may include groups of negatively charged microbubbles and positively charged microdroplets permanently held together by their opposing electrostatic attractive forces to form a single, agglomerated entity.

### INCORPORATION BY REFERENCE TO ANY PRIORITY APPLICATIONS

Any and all applications for which a foreign or domestic priority claim is identified in the Application Data Sheet as filed with the present application are hereby incorporated by reference under 37 CFR 1.57.

### BACKGROUND OF THE INVENTION

### Field of the Invention

### Tissue Ablation Therapy

In tissue ablation therapy, layers of tissue are locally destroyed by subjection to a destructive environment. Ultrasound is well suited to ablation therapy since it can produce both mechanical and thermal energy in a targeted region and may deliver this energy through pressure waves.

### HIFU

High Intensity Focused Ultrasound (HIFU) induces a destructive effect to achieve direct or indirect cell death in a limited target tissue volume. There are two mechanisms of action of HIFU: a thermal ablation mechanism and a mechanical tissue destruction mechanism. The thermal effect may be hyperthermia, wherein tissue temperatures are elevated. In particular, the thermal effect of HIFU is heat generation due to absorption of the acoustic energy with a rapid elevation of temperature in the local tissue leading to instantaneous and irreversible cell death via coagulation necrosis. The mechanical effect is cavitation wherein the HIFU sound field interacts with a gas bubble in the targeted tissue. Cavitation refers to a range of complex phenomena that involve the creation, oscillation, growth and collapse of bubbles within a medium.

The efficacy of HIFU for tissue ablation is dependent on the frequency of the HIFU field, and the depth of the target tissue region. For target tissue that is far from the HIFU source, or located such that there is a strongly attenuating medium between the HIFU source and the target tissue, the HIFU must have a lower frequency to be effective, for example a frequency less than 1 MHz. Examples of such target tissue include target tissue inside the skull or target tissue in the central abdomen such as the pancreas or certain regions of the liver. For target tissue that is located superficially on the body, or closer to the HIFU source, the optimal frequency is higher, for example a frequency greater than 10 MHz.

The HIFU treatment volume length can be approximated as inversely proportional to the HIFU frequency, and inversely proportional to the HIFU source's physical size. Therefore, to achieve efficient thermal ablation with HIFU in target tissues that are far from the HIFU source, or located such that there is a strongly attenuating medium between the HIFU source and the target tissue, and have a small thickness, the HIFU source must have a large physical size to reduce the risk of adverse effects that are associated with depositing too much energy to a volume of surrounding healthy tissue.

### HIFU With Microbubble Technology

Increasing the acoustic intensity and/or prolonging sonication duration can change a treatment outcome, such as a more efficient destruction of a larger volume of a target tumour. However, if the acoustic intensity is too high, there is a risk of depositing too much energy to a volume of healthy tissue surrounding the target and/or proximate the HIFU source. This may lead to adverse effects.

Primarily, thermal technologies such as HIFU have previously been combined with microbubble technology to reduce damage to surrounding healthy tissue. Examples of microbubble technology are preformed lipid-coated microbubbles which have predominantly been developed for ultrasound imaging. The lipid-coated microbubbles may be a contrast agent for ultrasound imaging due to their compressible gas cores, which render the microbubbles echogenic, and strongly absorbing, in a certain frequency band that is dependent on the microbubble diameter. When insonated with a HIFU field in this frequency band, the microbubbles absorb energy from the HIFU field more efficiently than the surrounding target tissue, and thus can lower the acoustic intensity threshold for ablation, thereby minimizing the thermal build-up of heat in surrounding tissue that can be associated with HIFU.

Microbubbles may induce additional heating of the target region by oscillation and cavitation, as well as generating shock waves, which delivers additional thermal energy to HIFU alone. Therefore, the addition of microbubbles proximate to the HIFU target region can enhance the ablation effect.

The microbubbles can be injected into a subject and travel through the subject's circulatory system until proximate to a target site. The method may lower the energy deposition required to achieve tissue ablation, shortening and optimising treatment time and incidence of adverse events.

However, use of regular microbubbles with thermal ablation techniques has several limitations. A first limitation is that the microbubbles have a short circulation time of approximately 2 to 3 minutes. HIFU takes a significantly longer time to set up and execute. The microbubbles are likely to have dissolved and/or dissipated before the HIFU process has been implemented. Another limitation is that these small microbubbles do not couple efficiently to low frequency ultrasound, for example the low frequency ultrasound used for transcranial applications. Yet further limitations are that the microbubbles have low spatial selectivity, have a residence time on the order of seconds in the target tissue, and may cause unintended heating away from the target site.

Hence, due to the above-mentioned limitations of HIFU, and HIFU in combination with diagnostic microbubbles, the limitations of ultrasound ablation technology remain unsolved.

The present application solves these above limitations by combining ultrasound technology with Acoustic cluster therapy (ACT)®.

### SUMMARY OF THE INVENTION

According to one aspect of the invention, there is provided a method of enhanced ultrasound ablation comprising: creating at least one ablation-assisting bubble proximate to a target region by: administering a cluster composition, comprising a microbubble component and a microdroplet component, to a subject, wherein the cluster composition comprises at least one cluster; and activating a phase shift transition of the microdroplet component of the at least one cluster by ultrasound insonation to create the at least one ablation-assisting bubble; wherein an expansion from the transition of the at least one cluster to the at least one ablation-assisting bubble provides a mechanical stress on the target region to aid ablation on target tissue in the target region.

The at least one ablation-assisting bubble may have a diameter of at least 10 micrometers.

The method may further comprise insonating the at least one ablation-assisting bubble with ultrasound at a predetermined intensity to induce at least one of energy absorption, deposition, oscillation and cavitation of the ablation-assisting bubble to provide an additional mechanical and/or thermal stress on the target region, to aid ablation on the target tissue in the target region.

The predetermined intensity of the ultrasound for insonating the ablation-assisting bubble may be equal to an intensity for non-bubble assisted ultrasound ablation divided by a factor of between 12 and 24.

The enhanced ultrasound ablation may be configured to provide a resultant temperature in the target region of 30 to 70 degrees Celsius, for a continuous exposure time of at least 30 seconds.

The method may further comprise using the at least one ablation-assisting bubble for real-time imaging of the target region by insonating the at least one ablation-assisting bubble with imaging ultrasound and wherein the at least one ablation-assisting bubble is induced as a hyperechoic spot.

The method may further comprise a further step of ultrasound planning comprising using unique subject and application specific information to plan a specific ultrasound exposure regime wherein ultrasound planning comprises at least one of a passive ultrasound planning and an active ultrasound planning.

Passive ultrasound planning may be based on one or more of: physiological information, anatomical structures in an ablation zone, cross-modality imaging and co-registration, and data defining subject anatomy retrieved from a software.

Active ultrasound planning may comprise: monitoring the ablation zone during the enhancement step for real-time feedback of the ablation zone; and adjusting the specific ultrasound exposure regime to achieve predetermined parameters in the ablation zone; wherein the steps of monitoring and adjusting are performed on a continuous basis for a predetermined duration.

Monitoring the ablation zone during the enhancement step for real-time feedback of the ablation zone may comprise at least one of: real-time temperature feedback, real-time mechanical feedback, cross-modality imaging and co-registration, real-time monitoring of cluster dynamics and concentration, and real-time monitoring of ablation-assisting bubble dynamics and concentration.

Monitoring the target region, including an ablation zone, may further comprise imaging via image guidance modalities, wherein the image guidance modalities comprise at least one of: magnetic resonance guided, ultrasound guided, computer tomography guided, optical guided, thermocouple guided, and contrast-enhanced ultrasound guided.

The method may further comprise co-administration of a therapeutic agent configured to assist ablation efficacy, the therapeutic agent pre-, and/or co- and/or post administered separately to the cluster composition.

According to a second aspect of the invention, there is provided an intravenously administrable composition for use in a method of enhancing ablation at a target region, the composition comprising: a microbubble/microdroplet cluster composition which forms at least one cluster via electrostatic forces; and wherein, when the method comprises exposing the at least one cluster to an effective exposure of ultrasound, each of the at least one clusters is configured to oscillate, expand and fuse into a single entity providing an ablation-assisting bubble.

When the method further comprises exposing the resulting ablation-assisting bubble to ultrasound at a predetermined intensity, said ablation-assisting bubble may be configured to oscillate and/or cavitate to induce mechanical and/or thermal stress on the target region to increase ablation efficiency in the target region.

The resulting ablation-assisting bubble may be configured to induce mechanical and/or thermal stress, comparable to an induced mechanical and/or thermal stress from direct ultrasound insonation on the target region, wherein the predetermined intensity is equal to an intensity for non-bubble assisted ultrasound ablation divided by a factor of between 12 and 24.

The microbubble/microdroplet cluster composition may be formed from: a cluster dispersion of microdroplets, having a median diameter of 2 to 3 μm, stabilised with a lipid membrane with a net positive surface charge; and microbubbles, having a median diameter of 2 to 3 μm, and stabilised with a lipid shell with a net negative surface charge.

The net positive surface charge of the microdroplets and the net negative surface charge of the microbubbles may provide the electrostatic forces which enable the at least one microbubble/microdroplet cluster to be formed.

The resulting formed clusters may have a diameter in the range of 4 to 8 μm.

A gas of the microbubbles of the at least one microbubble/microdroplet cluster may comprise sulphur hexafluoride or a C3-6 perfluorocarbon or mixtures thereof.

An oil phase of the microdroplet of the at least one microbubble/microdroplet cluster may comprise a partly or fully halogenated hydrocarbon or a mixture thereof.

The composition of the second aspect of the invention may be for use in treatment of one or more of tumours, space occupying masses, thrombolysis and neurological diseases.

### BRIEF DESCRIPTION OF THE DRAWINGS

FIG. 1 shows a flowchart of a method for ACT® enhanced ultrasound ablation according to the invention;

FIG. 2 shows a step wise diagram of the method for ACT® enhanced ultrasound ablation;

FIG. 3 shows a graph of a number of generated ablation-assisting bubbles against mechanical index at different frequencies;

FIG. 4a is a schematic diagram of a first example transducer having a relatively low operating frequency and a relatively large aperture;

FIG. 4b is a schematic diagram of a second example transducer having a relatively high operating frequency and a relatively large aperture;

FIG. 4c is a schematic diagram of a third example transducer having a relatively low operating frequency and a relatively small aperture;

FIG. 4d is a schematic diagram of a fourth example transducer having a relatively high operating frequency and a relatively small aperture;

FIG. 5a is a schematic diagram of a first example combination of the transducers of FIG. 4a to FIG. 4d;

FIG. 5b is a schematic diagram of a second example combination of the transducers of FIG. 4a to FIG. 4d;

FIG. 5c is a schematic diagram of a third example combination of the transducers of FIG. 4a to FIG. 4d;

FIG. 5d is a schematic diagram of a fourth example combination of the transducers of FIG. 4a to FIG. 4d;

FIG. 6a is a schematic diagram of a further example configuration of transducers;

FIG. 6b is a schematic diagram of a further example configuration of transducers;

FIG. 7a is a schematic diagram of a further example configuration of transducers;

FIG. 7b is a schematic diagram of a yet further example configuration of transducers;

FIG. 8a is a graphical representation of calculations of the maximum differential volume of ablation assisting bubbles oscillating in a first mode of oscillation in a free field with a mechanical index of 0.4;

FIG. 8b is a graphical representation of calculations of the maximum differential volume of ablation assisting bubbles oscillating in a first mode of oscillation in a free field with a mechanical index 0.6; and

FIG. 8c is a graphical representation of calculations of the maximum differential volume of ablation assisting bubbles oscillating in a first mode of oscillation in a free field with a mechanical index of 0.8.

### DEFINITIONS

Unless otherwise defined, all terms of art, notations and other scientific terms or terminology used in this text are intended to have the meanings commonly understood by those of skill in the art to which this invention pertains. In some cases, terms with commonly understood meanings are defined herein for clarity and/or for ready reference, and the inclusion of such definitions herein should not necessarily be construed to represent a substantial difference over what is generally understood in the art.

As used herein, ‘subject’ means any human, or non-human animal individual selected for treatment or therapy, and encompasses, and may be limited to, a patient, particularly to a human patient.

‘Insonation’ or ‘ultrasound insonation’ are terms used to describe exposure to, or treatment with, ultrasound.

The term frequency is defined as number of (ultrasound) cycles per second (Hz). When used herein the term designates the centre frequency of the applied sound field.

The term ‘regular medical imaging ultrasound’ is used to describe ultrasound from commercially available ultrasound scanners and probes intended for medical imaging.

The term ‘High intensity Ultrasound’ or ‘HIFU’ is used to describe ultrasound at an intensity which exceeds diagnostic limits.

The term ‘microdroplet’ is used to describe emulsion microdroplets with a diameter in a range from 0.2 to 10 μm.

The term ‘microbubble’ or ‘regular, contrast microbubble’ is used to describe gas bubbles with or without a stabilising shell having a diameter in a range from 0.2 to 10 μm, typically with a mean diameter between 2 to 3 μm. ‘Regular, contrast microbubbles’ include commercially available agents such as Sonazoid® (GE Healthcare), Optison® (GE Healthcare), Sonovue® (Bracco Spa.), Definity® (Lantheus Medical Imaging), Micromarker® (VisualSonics Inc.) and Polyson L® (Miltenyi Biotec GmbH).

The terms ‘microbubble/microdroplet cluster composition’, ‘microbubble and microdroplet cluster composition’, or ‘cluster composition’ refer to a composition having a first component of microbubbles and a second component of microdroplets, in particular oil microdroplets.

The term ‘clustering’ refers to a process where microbubbles in the first component and microdroplets of the second component form clusters.

The term ‘cluster’ refers to groups of microbubbles and microdroplets in the cluster composition permanently held together by electrostatic attractive forces, in a single agglomerated entity.

The term ‘phase shift’ is used to describe the phase transition from the liquid to gaseous states of matter. Specifically, the transition (process) of the change of state from liquid to gas of an oil component of microdroplets within a cluster upon ultrasound insonation.

The term ‘activation’ or ‘activation step’ refers to the induction of a phase shift of microbubble/microdroplet clusters by ultrasound insonation.

The terms ‘ablation assisting bubbles’ or ‘AA bubbles’ in this text is used to describe large (>10 μm) bubbles that form after ultrasound induced activation of the cluster (i.e., bubbles resulting from the ‘activation step’).

The term ‘enhancement’ or ‘enhancement step’ refers to the induction of volume oscillations and/or cavitation of the ablation-assisting bubbles and ensuing biomechanical effects, by ultrasound insonation.

‘Acoustic Cluster Therapy(®)’ or ‘ACT(®)’ refers to the process of administering a cluster composition to a subject, activating a phase shift of at least one resulting cluster by ultrasound insonation to generate ablation-assisting bubbles, and using the ablation-assisting bubbles in a further enhancement step.

### DETAILED DESCRIPTION OF THE PREFERRED EMBODIMENT

The invention provides a method of enhanced ablation therapy (ACT® enhanced ablation therapy), in particular a method of enhanced ultrasound ablation (ACT® enhanced ultrasound ablation therapy).

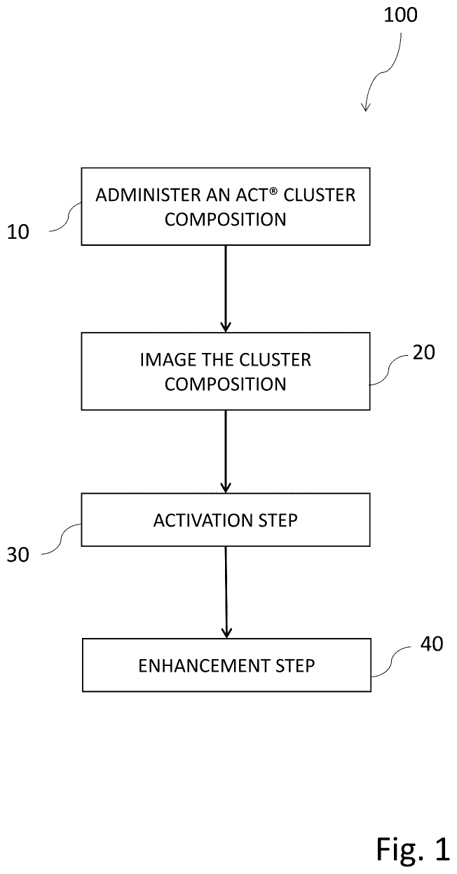

With reference to FIG. 1, Acoustic Cluster Therapy (ACT)® enhanced ablation therapy comprises:
(i) administering 10 a cluster composition to a subject, the cluster composition forming one or more clusters wherein microbubbles and microdroplets are permanently held together by electrostatic attractive forces;
(ii) optionally, imaging 20 the one or more clusters using ultrasound imaging to identify a region of interest (target region) for treatment within said subject;
(iii) an activation step 30 comprising activating a phase shift of a diffusible component of the microdroplet of the cluster from step (i) by ultrasound insonation at an activation frequency, and optionally an activation mechanical index, to form at least one ablation-assisting (AA) bubble in the target region;
(iv) optionally, an enhancement step 40 comprising insonating the at least one AA bubble with ultrasound to cause oscillations and/or cavitation of the at least one AA bubble;
(v) optionally, a monitoring step comprising monitoring the effect of the insonation during the enhancement step (iv) and adjusting at least one ultrasound parameter in accordance with a suitable metric, for example an ablation efficacy, a temperature in the target region, and/or bubble oscillation dynamics in the target region; and
(vi) optionally, an iteration step wherein steps (iv) and (v) are repeated for a prescribed time period.

### Administration

The cluster composition is administered to a subject parenterally, preferably intravenously. The cluster composition forms one or more agglomerated cluster due to attractive electrostatic forces of the composition components. The one or more clusters may be formed before or after administration in the subject. Preferably, the one or more clusters is formed before administration in the subject.

In an example, the microbubble and microdroplet cluster composition is formed from a cluster dispersion of microdroplets (second component) stabilised with a lipid membrane with a net positive surface charge, and microbubbles (first component) stabilised with a lipid shell with a net negative surface charge. In an example, both the microdroplets and microbubbles have a median diameter of 2 to 3 μm. The net positive surface charge of the microdroplets and the net negative surface charge of the microbubbles provides the electrostatic forces which enable the at least one cluster to be formed.

In an example, the first component comprises a dispersed gas selected from the group of sulphur hexafluoride, perfluoropropane, perfluorobutane, perfluoropentane and perflurohexane or a mix thereof, stabilized by a first stabilizer selected from the group of phospholipids, proteins and polymers. The second component comprises a diffusible component selected from the group of perfluorocarbons, for example, a perfluorocycloalkane, stabilized with a second stabilizer selected from the group of surfactants, for example including phospholipids, polymers and proteins. More specifically, either of the stabilizers are selected from phospholipids.

In a specific example, the first component consists of per-fluorobutane (PFB) microbubbles stabilized by a hydrogenated egg phosphatidyl serine-sodium (HEPS-Na) membrane and embedded in lyophilized sucrose. HEPS-Na carries a negatively charged head group with an ensuing negative surface charge of the microbubbles. Each vial of first component contains approximately 16 μL or 2·109 microbubbles, with a mean diameter of approximately 2.0 μm. The freeze-dried formulation displays long shelf life, more particularly 3 years, stored at ambient room temperature. In this specific example, the second component consists of perfluoromethyl-cyclopentane (pFMCP) microdroplets stabilized by a 1,2-Distearoyl-sn-glycerol-3-phosphocholine (DSPC) membrane with 3% mol/mol stearylamine (SA) added to provide a positive surface charge. The microdroplets in the second component are dispersed in 5 mM TRIS buffer. The standard formulation of the second component, in this specific example, contains approximately 4 μL or 0.8·109 microdroplets per mL, with a mean diameter of approximately 1.8 μm. The second component displays long shelf life, more particularly 18 months or more, stored refrigerated. The cluster composition may be prepared aseptically by reconstituting a vial of the first component with 2 mL of the second component followed by 30 seconds of manual homogenisation. 2 mL may be withdrawn from a vial of the second component using a sterile, single use syringe and needle. The content of the syringe can be added through the stopper of a vial of first component and the resulting cluster composition may be homogenised, preparing the composition for administration.

### Imaging

The method may include the optional step of using low mechanical index (MI) contrast agent imaging modes to image the microbubble component of the clusters to identify a pathological location for treatment. In said low MI contrast agent imaging modes the insonating ultrasound has an MI below 0.1. Standard medical ultrasound contrast agent imaging may be performed without triggering cluster activation since the clusters are not activated at low MI (below the activation threshold). Thus, the clusters can be used for imaging, for example, to identify tumour microvascular pathology before subsequent steps in the ablation method of the invention.

### Activation

After formation and administration of the clusters, the clusters are activated within, at or near the region of interest by application of ultrasound energy towards the region of interest and the target site. Alternatively, the clusters may be activated in feeding arteries outside the region of interest, depositing the activated bubbles in capillary beds downstream of the activation site, nearest to the region of interest. Thus, activated AA bubbles can be spatially localised in a tissue or organ of interest, such as proximate a tumour, by spatially localised application of the ultrasound energy to activate the clusters.

During the activation step 30, the cluster microbubbles oscillate and transfer energy to the cluster microdroplets. The oscillating microbubbles initiate an instant vaporisation (phase-shift) of the attached microdroplets, resulting in the formation of AA bubbles. Said AA bubbles transiently deposit (lodge) in the microvasculature of the subject. In particular, the activated AA bubbles transiently deposit in a nearest capillary bed down-stream of the site of activation, in an amount correlated to the blood perfusion of the tissue.

Since the resonance frequency of the microbubble component is typically in the range of 2 to 5 MHz, the clusters are readily activated by frequencies in the regular medical imaging range of 1 to 10 MHz with MIs above 0.1. However, an activation frequency of the clusters is application dependent and frequencies in the range of 50 kHz to 20 MHz are possible.

In an example, the clusters are activated with standard diagnostic ultrasound imaging pulses (1 to 10 MHz) which are typically used in conventional medical ultrasound. Preferably the ultrasound imaging pulses have an MI between 0.1 and 0.4, more preferably between 0.15 and 0.3.

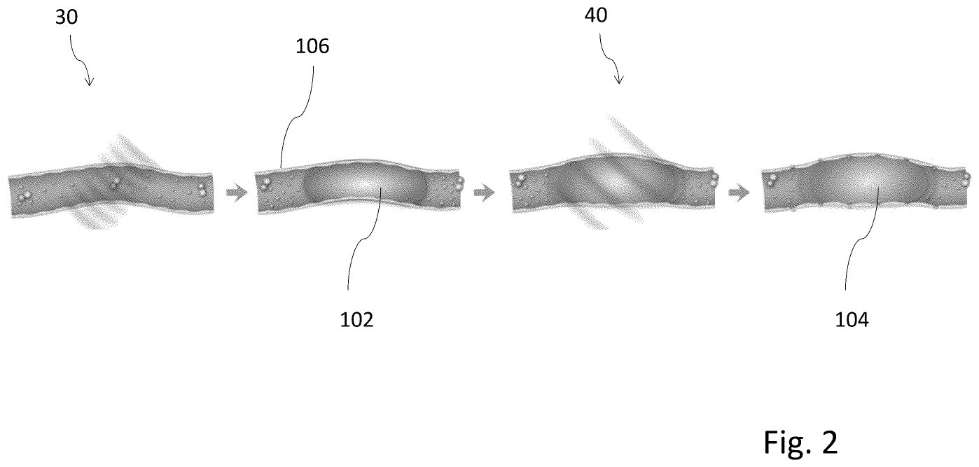

In one embodiment and with reference to FIG. 2, the activation step starts imminently after each administration of the cluster composition, such as within 20 seconds, and preferably lasts between approximately 60 to 120 seconds. The activation under medical ultrasound imaging control using the imaging pulses allows spatially targeted activation of the clusters in the tissue region being interrogated by the ultrasound field. After activation, the AA bubbles 102 produced are temporarily trapped in the microvasculature 106 of the targeted pathology due to their large size. The AA bubbles 102 are approximately 1000 times the volume of the pre-existing emulsion microdroplet prior to vaporisation. For example, a 20 μm diameter AA bubble may result from a pre-existing 2 μm diameter oil microdroplet. The activated bubbles (AA bubbles) are typically approximately 20 μm in diameter. The activated AA bubble may gradually shrink and advance further down in the capillary tree by intermittent lodging and dislodging, before it clears completely after typically 5 to 15 minutes.

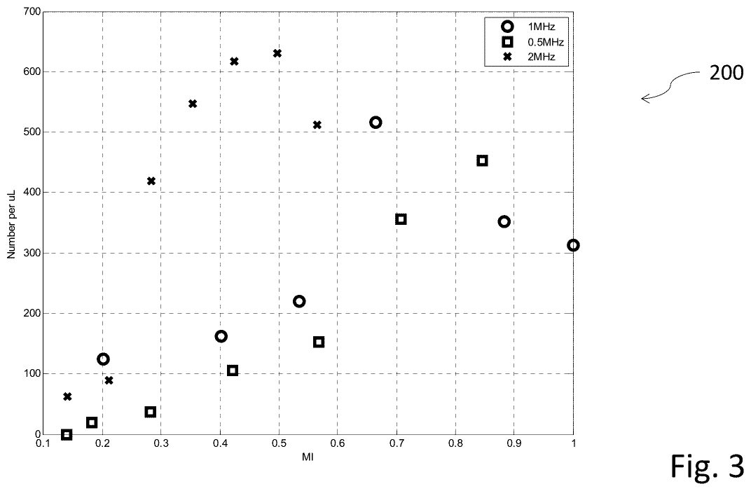

FIG. 3 shows a graph 200 of a number of successfully activated AA bubbles per μL against MI at three different frequencies. These frequencies are 0.5 MHz, 1 MHz and 2 MHz. The graph shows a peak number per μL of approximately 640 AA bubbles per μL when insonated with an ultrasound field having a frequency of 2 MHz and a MI of 0.5. Preferably, the number of generated AA bubbles per μL is greater than 300. Thus, according to the graph of FIG. 3, an ultrasound field having a frequency of 2 MHz and an MI between 0.29 and 0.6 is suitable. An ultrasound field having a frequency of 1 MHz is suitable with an MI of 0.65 and above. An ultrasound field having a frequency of 0.5 MHz is suitable with an MI of 0.7 and above.

### Enhancement

The enhancement step 40 comprises transferring ultrasound energy to the AA bubble to induce at least one of energy absorption, deposition, oscillation and cavitation of the activated AA bubble to provide an additional mechanical and/or thermal stress on a target region, in addition to a mechanical and/or thermal stress derived directly from ultrasound insonation on the tissue which has the effect of increasing ablation efficiency of cells in the target region. The ablation frequency may be the same or different to the ultrasound frequency used in the activation step. In some examples, a pulse amplitude of the ultrasound associated with the enhancement step is different to that of the activation step.

With further reference to FIG. 2, ultrasound insonation induces controlled volume oscillations 104 of the activated AA bubbles, preferably until cavitation, thereby exerting biomechanical forces on tissue in the target region. Hence, it has been found that the application of ultrasound, at or close to a cavitation frequency of the AA bubbles, can be used to produce additional mechanical and/or thermal bio-effect mechanisms to increase ablation therapy efficacity alongside effects from focused ultrasound directly onto the tissue in the region of interest. This leads to increased targeted tissue destruction.

Ultrasound exposures are dependent on frequency, exposure time, transducer characteristics—for example geometry and configuration—total power delivered, acoustic pressure and intensity, and energy delivery mode. Specific ultrasound exposure regimes are selected based on the tissue type of the target region, the desired ablation effect and the ultrasound delivery path, as will be explain in more detail below.

Preferably the frequency of the insonated ultrasound at the enhancement stage is less than 3 MHz, and more preferably less than 1 MHz. However, the frequency may be outside this range, depending on the location of the target treatment volume in the subject, and the tissue characteristics of said volume. In the target volume, the target MI of the ultrasound field is preferably greater than 0.4, more preferably greater than 0.6, and even more preferably greater than 0.8.

The target time-averaged intensity of ultrasound used for ablation is in the range up to 5000 W/cm², depending on the specific application. With AA bubbles in the target tissue, the intensity required to achieve the same amount of ablation is reduced by a factor greater than 1, preferably greater than 8, more preferably greater than 16, furthermore preferably greater than 24, yet further preferably greater than 50, and yet further preferably greater than 100.

The enhancement step 40 is performed either non-invasively or invasively, and may be performed by a focused ultrasound array or a focused mono-element ultrasound transducer, or by one or more surgically implanted ultrasound transducers.

The method may comprise two distinct steps of insonation for the activation step and the enhancement step. Insonation may be discontinued after completion of the activation step before further insonation occurs for the enhancement step. Parameters of the ultrasound field may be modified for said further insonation. Alternatively, the ultrasound field provided for both insonation at the activation step and the enhancement step may be unchanged and have the same parameters (i.e., frequency, intensity, MI) throughout the ACT enhanced ablation therapy process. This is rendered possible since the use of AA bubbles reduces a required ultrasound power for achieving the desired ablation effect. This may be advantageous since it simplifies the process, for example, rendering only one transducer setup necessary.

The combination of ultrasound with AA bubbles have been shown to reduce the acoustic energy levels needed for ablation therapy by a factor of 100 or more. The ultrasound field interacts with the AA bubble in the form of acoustic cavitation. This causes the AA bubble to oscillate, grow and collapse. The acoustically driven AA bubble oscillations result in heat production, microstreaming of fluid near the bubble, and localized shear stresses. The absorption of energy as ultrasound propagates through a medium also produces a heating effect. In tissue, the rate of absorption increases with frequency. The AA bubbles may generate higher harmonics of the excitation frequency, further enhancing the heating effect.

### Ultrasound Planning

An optional ultrasound planning step may comprise a first passive ultrasound planning step and a second active ultrasound planning step.

In the first passive ultrasound planning step, unique subject and application specific information is gathered and processed to plan a specific ultrasound exposure regime. This information may include physiological information and anatomical structures in the ablation zone, and use anatomy determining software. The software to define anatomy enables treatment of different kinds of patients in order to deliver accurate thermal doses.

An example of a type of physiological information to be used in ultrasound planning is perfusion of different organs. Thermally sensitive organs, such as the diaphragm, bowel, and spinal cord, are anatomical structures of particular interest in the ablation zone when planning the ultrasound exposure.

In the second active ultrasound planning step, real-time feedback of conditions in the ablation zone can be used to adjust the ultrasound parameters in an iterative process to ensure optimum conditions throughout the ACT enhanced ultrasound ablation method. The real-time feedback may comprise: real-time temperature feedback, real-time mechanical feedback, cross-modality imaging and co-registration, real-time monitoring of the cluster dynamics and concentration, and real-time monitoring of the AA bubble dynamics and concentration.

Real-time mechanical feedback may be obtained by cavitation detection using ultrasound. Since the generated AA bubbles are highly echogenic, ultrasound backscatter from the AA bubbles themselves can be used in imaging for cavitation detection. Real-time mechanical feedback may also be obtained via shearwave monitoring (i.e., using elastography).

Real-time temperature feedback can be obtained using thermocouples, fibreoptic thermosensors, and Magnetic Resonance Imaging (MRI).

During the ablation process, ultrasound parameters may be modified based on the real-time feedback assessed against desirable ablation zone conditions. For example, an ablation efficacy, a temperature in the target region, and bubble oscillation dynamics in the target region may be modified based on the real-time feedback. Gathering of the real-time feedback (monitoring) and readjusting the ultrasound parameters accordingly can be implemented in a continuous feedback loop for the duration of the ablation treatment, or for a predetermine duration such as the duration of the enhancement step.

The optimal choice of ultrasound frequency is application-specific and represents a compromise between treatment depth and the desired rate of heating. Frequencies near 1 MHz have been found to be most useful for heat deposition, with frequencies as low as 0.5 MHz being used for deep treatments or with large absorption portion in the propagation path (transcranial application) and as high as 8 MHz for superficial treatments (e.g. prostate and melanoma).

A threshold thermal dose for achieving desired thermal effects including irreversibly damaged and coagulate critical cellular protein, tissue structural components and the vasculature leading to immediate tissue destruction varies with tissue type and exposure time. For normal tissue, the temperature range is between 30 to 77° C., whereas for tumour tissue the temperature range is between 41 to 64° C. For a majority of applications, the threshold thermal dose is in the region of 43 to 65° C. for a continuous exposure time of around 30 seconds.

### Monitoring

The ablation zone benefits from careful monitoring and provides the real-time feedback for the second active ultrasound planning step. As mentioned above, monitoring of the ablation zone may comprise monitoring the temperature, mechanics, cluster and bubble dynamics and concentrations. Various methods can be employed for monitoring the ablation zone.

A particular method of monitoring the ablation zone is using image guidance modalities. Example of image guidance modalities comprise magnetic resonance (MR) guided, ultrasound guided, computer tomography (CT) guided, optical guided, thermocouple guided, and contrast-enhanced ultrasound guided, or combinations of the above.

The scattering cross sections of AA bubbles are orders of magnitude greater than the scattering cross sections of the micron sized microbubbles comprised in the clusters before activation. As a result, the AA bubbles produce copious backscatter signals and are readily imaged in fundamental imaging mode with diagnostic imaging systems. The resonance frequencies of the AA bubbles are also an order of magnitude lower (approximately 0.2 to 0.8 MHz) than the resonance frequencies of the microbubbles comprised in the clusters before activation.

MRI guided ablation therapy has excellent anatomical resolution and, in particular, a high sensitivity for tumour detection, thereby offering accurate planning of the tissue to be targeted. In order to be used in the high magnetic fields of MRI, ultrasound transducers must be specially designed for compatibility. MR guided ultrasound has the additional benefit of providing soft tissue contrast, quantitative thermometry, temperature feedback control, diagnostics, but may have limited accessibility. MRI is very sensitive to temperature changes such that ongoing, real-time feedback of thermal data can be provided throughout the procedure. MRI is particularly suitable to ACT enhanced ablation therapy in which bio-mechanical effects compensate for thermal effects and, as such, temperatures generated from the process may be relatively low in ablation terms.

Ultrasound guided ablation therapy is widely accessible, has good temporal resolution, provides soft tissue contrast and diagnostic, and provides qualitative feedback. An ultrasound diagnostic transducer is usually incorporated into the treatment head which allows real-time imaging of the ablation process.

Magnetic Resonance-guided Focused Ultrasound (MRgFUS) is a type of MRI guided ablation therapy. During the MRgFUS procedure, the patient is conscious, and the functional effects of the procedure are clinically assessed throughout. The operator continuously controls and refines the region of interest (target) and choses the attributes of the incident ultrasound field with regards to level of MI and number of insonations.

Median duration in the MRgFUS procedure is about 1 hour, which includes acquisition of planning sequences, targeting and sonication. Repeated injection or infusion of the ACT microcluster composition can be made to cover the MRgFUS procedure time span if required.

### Hardware

ACT enhanced ultrasound ablation may use one or more ultrasound transducers or transducer arrays for providing the ultrasound fields for the activation step and, optionally, the imaging, enhancement, and monitoring steps of the procedure.

An ultrasound probe provides the ultrasound field for insonation at the activation step and the enhancement step. The use of AA bubbles may negate the need for HIFU, so the ultrasound probe may comprise a commercially available transducer that is not designed specifically for HIFU applications.

An extracorporeal ultrasound device is usually used for targets lying within the breast, abdomen, brain or limbs. Transcutaneous treatments require an appropriate acoustic window on the entry site that provides a propagation path for the focused ultrasound beam that is uninterrupted by intervening gas.

In one embodiment, an external (non-invasive) transducer is used. Such an external transducer provides the opportunity of combining the ACT technology with MRI guided focused ultrasound (MRgFUS).

In an example, the selected ultrasound transducer emits ultrasound waves at a frequency in the range of 1 to 5 MHz focal intensities, and approximately −6 dB beam size, wherein a −6 dB beam size is approximately 1 to 3 mm in width and 10 mm in length depending on geometrical size and acoustic parameters.

The ultrasound transducer may be a stationary transducer which provides a single exposure and is suitable for target regions of small volumes. The volume of the target region that is treatable by the stationary transducer is dependent on the particular frequency and geometry of the transducer, and also the location of the target region in relation to the transducer. The treatable volume may be approximated as an ellipsoid having principle diameters A, B, C, so that the treatment volume is approximately:

$$V = \frac{π}{6} ABC$$

Example values for the principle diameters A and B are 1 to 3 mm, and an example value for the principle diameter C is 10 mm. Thus, examples of treatment volumes are in the range of 5 to 50 mm³.

The ultrasound probe may also comprise an ultrasound transducer which provides the ability to direct the ultrasound field towards multiple target regions by means of one of: physically rotating and/or translating the transducer array within the ultrasound probe housing; or electronically exciting specific transducer elements in a specific sequence. Thus, the probe is suitable for target regions of larger volumes, for example several times the volume range for stationary transducers.

The probe may be combined with a catheter or in an enclosed appendage for intracorporeal administration of ultrasound, for example when the target volume is in the prostate and the probe is inserted into the urinary tract or the rectum. Other applications may call for the use of such a device into a suitable orifice such as the vaginal tract, nasal cavity, mouth, or oesophagus.

The probe may comprise a therapeutic transducer which is designed to deliver ultrasound with a higher power and relies on the same principles as conventional ultrasound transducers.

A probe may comprise two or more transducers or array transducers that operate at independent frequencies, wherein a first set of transducers or transducer arrays provide the insonation at the activation step, and a second set of transducers or transducer arrays provide the insonation at the enhancement step.

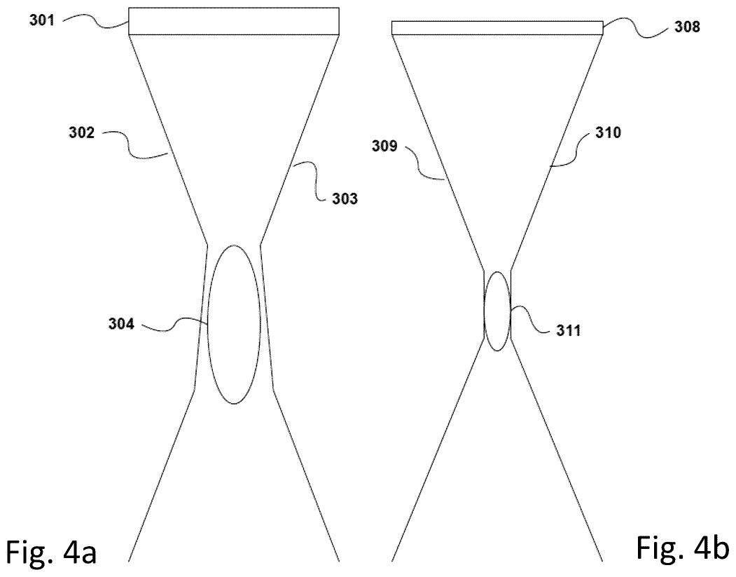

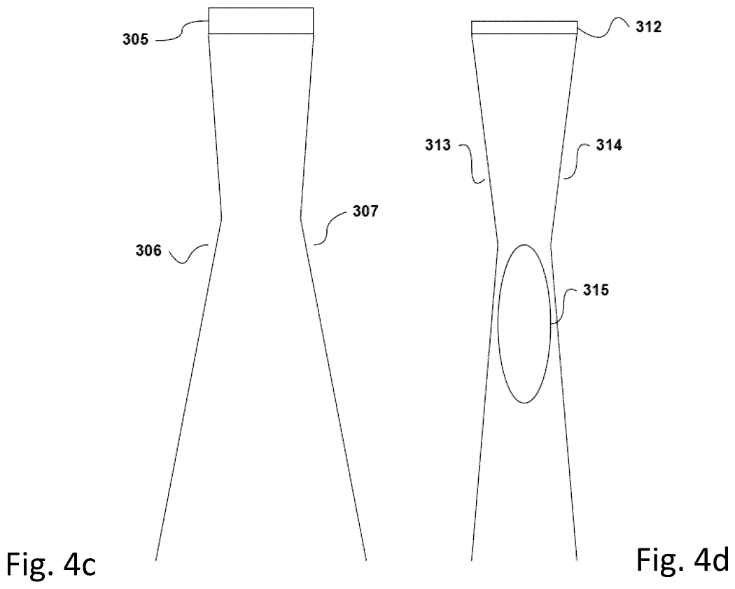

FIG. 4a is a schematic diagram of a first example transducer 301 having a relatively low operating frequency, for example around 1.25 MHz, and a relatively large aperture, for example around 50 mm. A line 302 indicates the extent of a transmitted ultrasound beam to the left, and a second line 303 indicates the extent of a transmitted ultrasound beam to the right. An oval shape 304 indicates a region where the intensity of the transmitted ultrasound beam is highest.

FIG. 4b is a schematic diagram of a second example transducer 308 having a relatively high operating frequency, for example around 2.5 MHz, and a relatively large aperture, for example around 50 mm. A first line 309 indicates the extent of a transmitted ultrasound beam to the left, and a second line 310 indicates the extent of a transmitted ultrasound beam to the right. An oval shape 311 indicates the region where the intensity of the transmitted ultrasound beam is highest.

FIG. 4c is a schematic diagram of a third example transducer 305 having a relatively low operating frequency, for example around 1.25 MHz, and a relatively small aperture, for example around 25 mm. A first line 306 indicates the extent of a transmitted ultrasound beam to the left, and a second line 307 indicates the extent of a transmitted ultrasound beam to the right.

FIG. 4d is a schematic diagram of a fourth example transducer 312 having a relatively high operating frequency, for example around 2.5 MHz, and a relatively small aperture, for example around 25 mm. A first line 313 indicates the extent of a transmitted ultrasound beam to the left, and a second line 314 indicates the extent of a transmitted ultrasound beam to the right. An oval shape 315 indicates the region where the intensity of the transmitted ultrasound beam is highest.

The transducers 301, 305, 308, and 312, are depicted as planar, but may also have a curved shape.

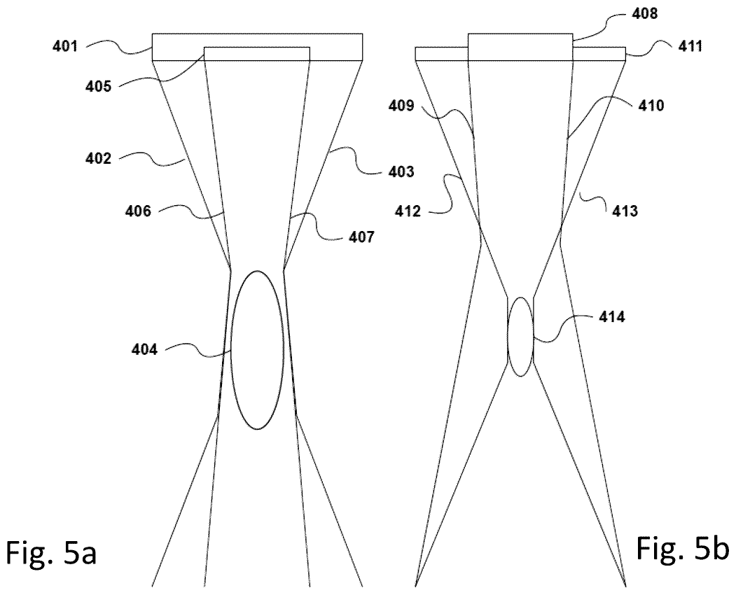

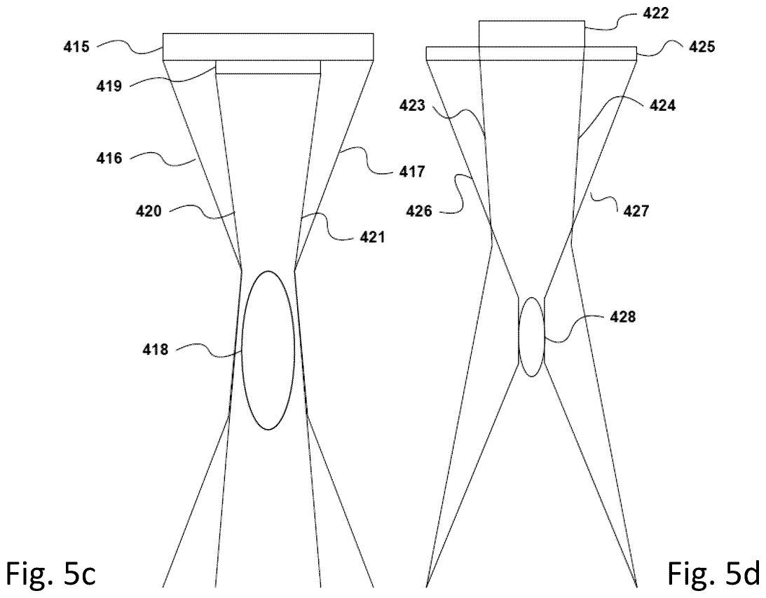

FIGS. 5a to 5d are schematic diagrams of examples of how transducers, similar to the transducers in FIGS. 4a to 4d, may be combined in a stacked or colocalized manner to achieve ablation suitable for specific applications.

In a first example shown in FIG. 5a, a relatively high frequency (RHF) transducer 405 is colocalized with a relatively low frequency (RLF) transducer 401. The RHF transducer has a smaller active aperture than the RLF transducer so that the transmitted ultrasound beam at lower frequency has an extent 402, 403, that is wider than the transmitted ultrasound beam at higher frequency 406, 407. In this example, the higher and lower frequency ultrasound beams have overlapping and equal regions of maximum intensity 404. This configuration may be suitable for applications where the target treatment volume is in the region of 10 to 500 mm³ and at a depth of around 3 to 7 cm.

In a second example shown in FIG. 5b, a RHF transducer 411 is colocalized with a RLF transducer 408, and the RHF transducer 411 has a larger active aperture than the RLF transducer 408 so that the transmitted ultrasound beam at higher frequency has an extent 412, 413, that is wider than the transmitted ultrasound beam at lower frequency 409, 410. In this example, the higher frequency ultrasound beam has a region of maximum intensity 414 that is suitable for activation in target volumes in the region of approximately 1 to 50 mm³ at a depth of approximately 1 to 4 cm. In this example, the lower frequency beam is used for enhancement in a region 414 determined by the higher frequency beam, even though the extent of the lower frequency beam is larger.

In further examples shown in FIGS. 5c and 5d, RLF transducers 415, 422 are vertically stacked with RHF transducers 419, 423, respectively. The same principles for activation and enhancement apply as in the previous example transducer setup.

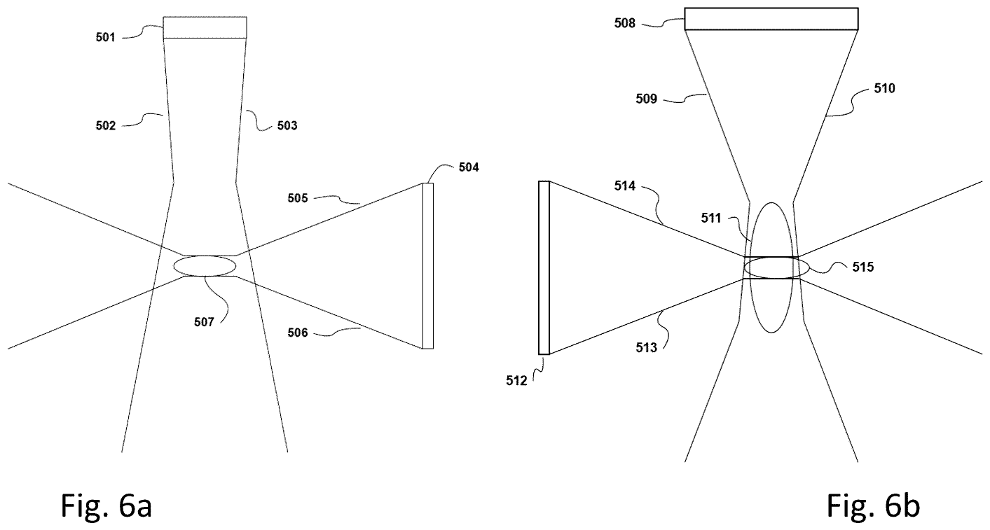

FIGS. 6a and 6b are schematic diagrams of two further example transducer configurations, wherein RLF transducers 501, 508 and RHF transducers 504, 512 are not colocalized or stacked.

The example of FIG. 6a shows a RLF transducer, having an unfocused ultrasound beam 502, and a RHF transducer 504 transmitting an ultrasound beam delimited by lines 505, 506 with a region of maximum intensity 507. In this example, the target tissue is to be located in the region 507.

In the example of FIG. 6b, a RLF transducer 508 is configured to transmit a focused ultrasound beam delimited by lines 509, 510, and provides a region of maximum intensity 511. A RHF transducer 512, is configured to transmit ultrasound beams delimited by lines 513, 514 and provides a region of maximum intensity 515. The target tissue is to be located in the overlap of the regions 511 and 515, with activation being provided by either the RHF transducers 512 or the RLF transducers 508, and enhancement being provided either the RHF transducers 512 or the RLF transducers 508.

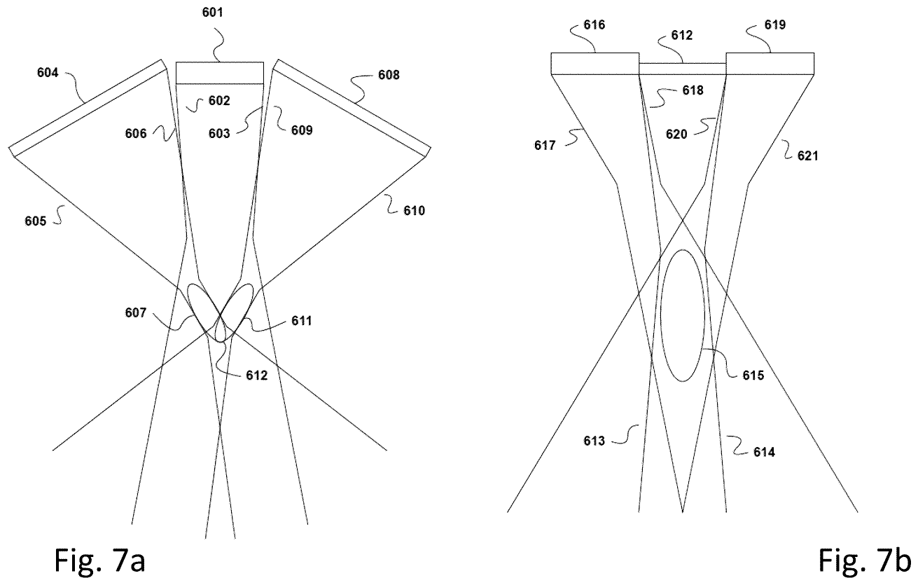

FIGS. 7a and 7b are schematic diagrams of two further examples of transducer configuration.

In a first example according to FIG. 7a, two RHF transducers 604, 608 are configured to transmit ultrasound beams with an extent 605, 606, 609, 610, respectively. RHF transducers 604, 608 provide respective regions of maximum intensity 607, 611 that overlap to form a region of combined maximum intensity 612. Placement of the RHF transducers 604, 608 is such that the region of combined maximum intensity 612 coincides with a transmitted ultrasound beam delimited by lines 602, 603 from a RLF transducer 601. The target volume is defined by the region of combined maximum intensity 612. Activation is provided by one of the RHF transducers 604, 608 or the RLF transducer 601. Enhancement is provided by one of the RHF transducers 604, 608 or the RLF transducer 601.

In a second example according to FIG. 7b, two RLF transducers 616, 619 configured to transmit respective ultrasound beams with an extent 617, 618, 620, 621 are placed so that the beams coincide with a region of maximum intensity 615 in an ultrasound beam 613, 614, transmitted by a RHF transducer 612. The target volume is defined by the region of maximum intensity 615. Activation is provided by the RHF transducer 612 or both of the RLF transducers 616, 619, and enhancement is provided by the RHF transducer 612, or both of the RLF transducers 616, 619.

A general system for implementing ACT enhanced ablation is a computer-controlled system and is suitable for generating ultrasound waves to achieve insonation of AA bubbles in the target region to achieve the activation step and, subsequently, the enhancement step as described above. The computer-controlled ACT enhanced ablation system may also utilise planning and treatment feedback.

A system for implementing ACT enhanced ablation can further comprise one or more of: a power amplifier, a pulse generator, a 3D positioning system and an imaging modality such as the US, CT, or MRI imaging modalities described above.

### Advantages

AA bubbles exhibit several characteristics which are different to regular microbubbles, and which enable ablation therapy to be performed at considerably lower ultrasound energy. A first characteristic it that the residence time of AA bubbles lodged in place in a capillary is approximately 5 to 15 minutes, in comparison to regular microbubbles, which do not lodge in place and pass through the capillary at a rate that is determined by the perfusion rate. This is typically in the order of a few seconds, depending on the tissue perfusion and volume. This increases a potential exposure time of the target region to the ACT enhanced ablation therapy. It is a major advantage to have a stationary bubble as the ultrasound procedure usually takes some time to perform (typically, ultrasound ablation is performed in the region of one hour), which a free-flowing bubble does not allow for. A second characteristic is that AA bubbles are large which enables increased generation of thermal and mechanical effects using lower acoustic power, in contrast to regular contrast microbubbles which are small, and thus the generation of thermal and mechanical effects is limited.

AA bubbles will also dissipate energy as heat due to friction between the surface area of the lodged AA bubbles and the capillary wall as well as via conduction during compression. Hence, the acoustic energy levels needed can be effectively reduced by combining ultrasound with AA bubbles.

The AA bubbles are activated and deposited in the tissue microvasculature under imaging control. Thus, spatial targeting of the AA bubbles in tissue is enabled. This, in combination with the AA bubble's prolonged residence time, allows more efficient and controlled implementation of ablation therapy.

The process is non-invasive and does not use any implantable hardware, thus this technique does not carry an infectious risk. It has the further benefit of not using ionizing radiation. Yet further it can provide an immediate effect.

Furthermore, AA bubbles can provide real-time imaging since they induce as hyperechoic spots. This gives rise to more information of the size and shape of the lesions wherein thermally ablated regions are not visible on B-mode ultrasound imaging.

It is often useful to assess perfusion of an organ to be treated before treatment is commenced. ACT technology can be used to test the perfusion of different organs thus providing a secondary utility alongside ablation enhancement, and a third alongside imaging (i.e., AA bubble accumulation at target tissue can be used for control feedback).

ACT enhanced ablation therapy may not reach such high temperatures, and has a reduced cooling time, in comparison to conventional thermal ablation. A cooling period between sonications is often required to prevent unwanted heating of surrounding tissue. Thus, less time between sonications is required leading to an overall reduction in ablation time.

All of the above outlined advantages may lead to the subsequent advantage of a reduced likelihood of side effects and haemorrhaging.

Thermal activity is enhanced only at an acoustic focus where pressure is sufficient for AA bubble activation. Ultrasound beam focusing enables high intensities only at a specific location within a small volume, which minimizes the potential for thermal damage to tissue outside the focal region. For example, the ultrasound beam may be approximately 1 mm in diameter and 10 mm in length. At the borders of the thermal coagulated lesion, the tissue will die within 2 to 3 days and be taken up by the immune system. Ultrasound used in combination with ACT enables lower intensities, achieving the same or better ablation, reducing the risk of damage to surrounding tissue.

### Utilities

ACT enhanced ablation has a clinical impact in neurology/surgery, ophthalmology, urology, gynaecology, and oncology. Within the field of neurology, ACT enhanced ablation may have a clinical impact on: brain tumours and space occupying masses; neuromodulation; tremor; tremor dominant Parkinson's disease (movement disorder symptoms); epilepsy; and stroke. Within the field of ophthalmology, ACT enhanced ablation may have a clinical impact on: glaucoma; intraocular tumours; retinal detachment; and trabeculotomies. Within the field of Urology, ACT enhanced ablation may have a clinical impact on: kidney stones; cervical pre-cancer lesions; and adrenal glands. Within the field of Gynaecology, ACT enhanced ablation may have a clinical impact on uterine fibroids and ovarian cancer. Within the field of Oncology, ACT enhanced ablation may have a clinical impact on: mainly musculoskeletal system; lung; breast; brain; prostate; kidney; liver; pancreas; brain tumours; renal; and vesical. ACT enhanced ablation may have a clinical impact on the following other malignancies: adrenal neoplasms; thyroid carcinomas; skin carcinomas; minor role in treatment of bulky tumours (neck, nodules, bones) and superficial (skin). Within the field of Cardiovascular disorders (block irregular electrical signals and restore typical heartbeat), ACT enhanced ablation may have a clinical impact on: atrial fibrillation; irregular heartbeat; arrhythmia; and normalization of blood vessel function. ACT enhanced ablation may also have a clinical impact on Pain treatment: palliative (metastasis); and chronic. Psychiatric disorders may also be treated with ACT enhanced ablation. Within the field of Cosmetic, ACT enhanced ablation may have a clinical impact on: signs of aging; and lifting. ACT enhanced ablation may also have a clinical impact on complete or partial vessel occlusions, for example deep venous thrombus, pulmonary embolism, coronary artery obstructions, and atherosclerosis.

Ultrasound settings (ultrasound exposure regimes) used in the enhancement step are application specific. In particular, application specific refers to tissue type to be ablated.

### ACT Enhanced Ultrasound Ablation to Treat Tumours

The temperature change is concentrated to a focal zone in and around the tumour. The overall objective of thermal tumour ablation is quite similar to that of surgery: remove the tumour and a 5 to 10 mm thick margin of seemingly normal tissue. In contrast to surgical removal which consists of physical excision, during thermal ablation, the tissue is killed in situ and then absorbed by the body over the course of several months.

Particular ultrasound settings for the enhancement step of ACT enhanced ultrasound wherein the target region is a tumour are: an MI of above 0.4, preferably above 0.6, and further preferably above 0.8; an intensity up to 5000 W/cm²; a frequency lower than 3 MHz and preferably below 1 MHz; and continuous or pulsed insonation for around 20 to 30 seconds.

ACT enhanced ultrasound ablation is suitable for the treatment of liver tumours and in the selective destruction of normal liver, bladder, muscle, and kidney. Depending on the equipment and parameters used, the volume of focused ultrasound lesions can be as small as a grain of rice (approximately 10 cubic millimetres). This allows for an extremely localized treatment and a sharp border between treated and untreated areas. For treatment of larger structures such as large tumours, multiple treatment volumes can be combined to encompass the entire volume.

Since tumours are metabolically active, they have a high perfusion compared to the relatively low perfusion of surrounding tissue. A higher perfusion rate of the tumour compared to the surrounding tissue ultimately results in a higher concentration of AA bubbles in the tumour tissue than the surrounding area. As described above, carrying out the enhancement step on the AA bubbles leads to destruction of the proximate tissue. Thus, ACT enhanced ultrasound ablation is particularly well suited to removing tumour cells since the technique can make use of the higher perfusion rate in tumours compared to normal tissue.

### ACT Enhanced Ultrasound Ablation for Signs of Aging

The particular ultrasound settings for the enhancement step of ACT enhanced ultrasound ablation, wherein the target region is signs of aging, are substantially similar to the settings for tumour ablation. However, the ultrasound field is preferably delivered in short bursts in place of continuous insonation.

Since the target region is normal tissue, the tissue should be subjected to a temperature within the temperature range of between 30 to 77° C.

### ACT Enhanced Ultrasound Ablation With Transrectal and Interstitial Ultrasound Sources

Transrectal and interstitial ultrasound sources can be placed closer to the target volume such that they may operate at lower powers and higher frequencies and achieve the same ablation efficacity. The preferred ultrasound power range for transrectal/interstitial sources during the enhancement step is up to 5000 W/cm². For the enhancement step, the preferred frequency is below 4 MHz, more preferably lower than 1 MHz. For larger prostates with deep lesions, the frequency selection is limited by required penetration depth.

ACT enhanced ultrasound ablation may be combined with chemotherapy, immune-therapy, and/or drug delivery to make treatment more effective and with fewer side effects. ACT enhanced ultrasound ablation does not exclude other therapeutic options. There is no negative cell selection when it comes to antibody or hormone therapy.

### ACT Enhanced Ultrasound Ablation for Treatment of the Brain

In the absence of combination with AA bubbles, the utility of thermal ultrasound ablation for treatment of the brain is limited, mainly due to the barrier of insonation through bone structure.

ACT enhanced ultrasound ablation has the advantage that the treatment time is reduced, the temperatures, and the frequency, can be kept lower than non-ACT enhanced ultrasound ablation so can overcome transcranial ultrasound limitations.

In addition, the AA bubbles may be close to the endothelial wall to allow for optimal generation of thermal effects since the bubbles are lodged in the vasculature for up to 15 minutes. This is in contrast to small regular contrast microbubbles, which have an average diameter of approximately 1 to 3 μm and thus, clear the vasculature in significantly less time, for example in the order of seconds, depending on tissue perfusion and volume.

A thickness of a skull varies with age, sex and across ethnic groups. Ultrasound ablation treatment is well suited to treatment through skulls having a thick barrier. Furthermore, ultrasound ablation may be limited to treatment of a target region at a centre of the brain. However, coupling the technology with an optimal AA bubble, enabling functionality with lower ultrasound energies, may resolve these issues. ACT provides the advantage is being transiently entrapped in the microcirculation of the region of interest. This combination makes it possible to fully utilize the MRgFUS technology, even working through thicker bone structures, for applications outside the centre in the brain, as well as providing opening of a blood brain barrier.

### ACT Enhanced Ultrasound Ablation to Treat Thrombolysis

ACT enhanced ablation, and ACT enhanced ultrasound ablation, can further be used for the treatment of the formation of a blood clot in the vasculature. The method can include the positioning of at least one cluster proximate, preferably adjacent, to a target blood clot formed in a subject's blood vessel. The cluster is then activated, according to the method described above, to create at least one AA bubble. The process of activation, wherein the cluster transitions into the AA bubble, results in an expansion of the entity which produces mechanical stress on the proximate (adjacent) blood clot. This mechanical stress may be adequate to cause a breaking up (breaking down) of said blood clot. The method of treating thrombolysis by breaking up blood clots may further comprise utilising an enhancement step of ACT enhanced ultrasound ablation such as the enhancement steps described above. The method of using ACT enhanced ultrasound ablation to treat thrombolysis may further comprise an activation step interleaved with an enhancement step alone or in combination with thrombolytic drugs or anticoagulants.

Generation of the AA bubble, and ACT enhanced ultrasound ablation, can provide fragmentation of the clot or reduce the clot size, promote motion of the clot, enhance the penetration of thrombolytics, anticoagulants into the clot, and/or remove clot degradation products by mechanical or thermal destruction.

ACT enhanced ultrasound ablation can be used as a treatment of Myocardial infarction, stroke, venous thromboembolism.

The ultrasound source used to provide the ultrasound field to achieve ACT enhanced ultrasound ablation to treat thrombolysis may be an external ultrasound probe or a catheter-based probe.

ACT enhanced ultrasound ablation to treat thrombolysis has the advantage that the risk of bleeding is reduced in comparison to breaking up of blood clots using ultrasound insonation alone, or using ultrasound insonation in combination with regular microbubbles. Bleeding can be reduced also since lower ultrasonic intensities are required. Bleeding may also be reduced since the method may not require, or required lower doses of, the use of thrombolytic drugs or anticoagulants. A further advantage is that treatment time can be reduced.

### EXAMPLES

### Simulations of enhancement field on bubbles

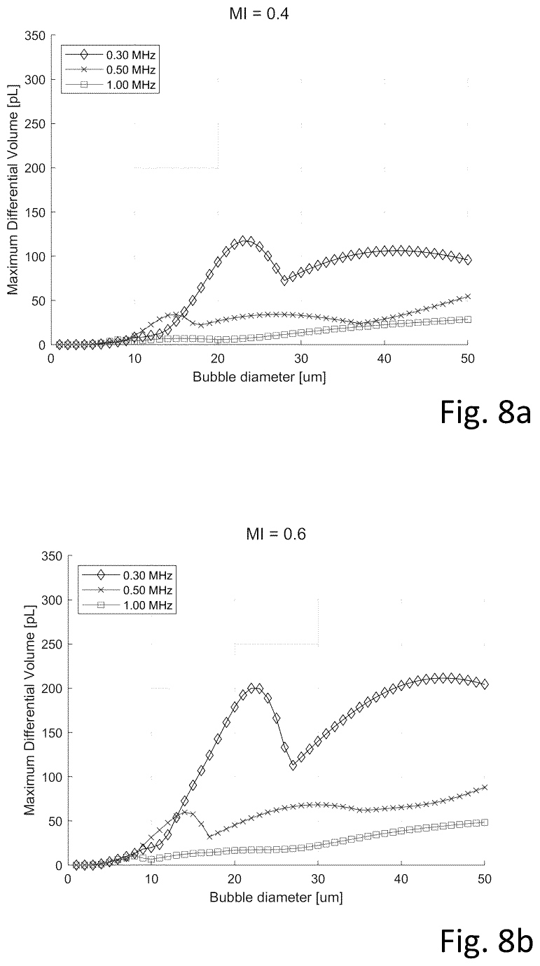

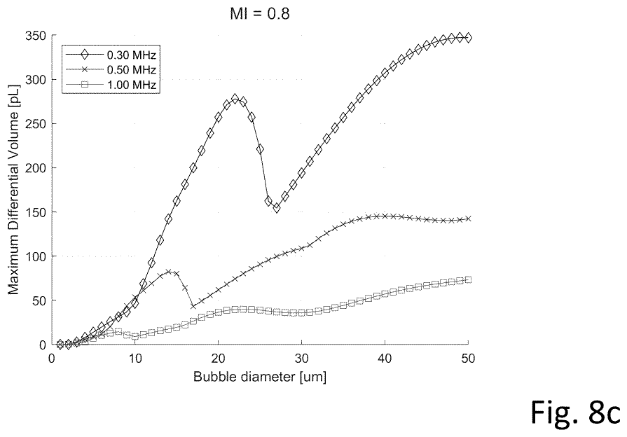

FIGS. 8a, 8b and 8c show graphical representations of calculations of the maximum differential volume of AA bubbles oscillating in a first mode of oscillation in a free field i.e., the difference between volume at peak expansion and volume at rest. The calculations are based on simulations of the modified Rayleigh-Plesset equation:

$$R \ddot{R} + \frac{3}{2} {\dot{R}}^{2} - \frac{{p}_{L} - {p}_{0} - {p}_{i}}{ρ} - \frac{{\dot{p}}_{L}}{ρ c} R = 0 ,$$

where R is the bubble radius, p_{L} is the pressure in the liquid at the bubble surface, p₀ is the ambient pressure, pᵢ is an incident driving pressure, p is the density of the surrounding fluid, and c is the speed of sound in the surrounding fluid. The variables c, p, and p₀ are constants, whereas pᵢ, P_{L} and R are functions of time, and the dot operator indicates differentiation with respect to time. The effect of bubble gas and shell parameters are contained within the surface pressure, p_{L}, which is a boundary condition for solving the equation for the radial oscillation, R. The number of cycles in the insonating pressure wave of the simulation is 2 cycles. The maximum differential volume is shown on the ordinate axis, and the AA bubble diameter is shown on the abscissa axis. FIGS. 8a to 8c reflect the maximum differential volume for different mechanical indexes of the insonating field. The mechanical index of FIG. 8a is 0.4, the mechanical index of FIG. 8b is 0.6, and mechanical index of FIG. 8c is 0.8. The frequency of the insonating field is 0.3, 0.5 and 1.0 MHz.as indicated by the legend in each of FIGS. 8a to 8c. These figures demonstrate the advantage of using lower frequency to achieve large volumetric oscillations of the AA bubble for larger AA bubble diameters. They also show that increasing the MI has the effect of increasing the maximum differential volume. For a higher MI, the AA bubble will undergo inertial cavitation, a process that causes gradual bubble size increase through inward diffusion of dissolved gases from the surrounding fluids and eventual violent bubble collapse. Stable and inertial cavitation will apply varying degrees of thermal and mechanical stress to the surrounding tissue.

### Pre-Clinical Evidence

Tumour-specific uptake of a fluorescent dye, Evans Blue® was investigated in a SC PC3 mouse model to investigate the effect of MI variance of the enhancement ultrasound field.

Three mice were selected to receive an enhancement ultrasound with a mechanical index of 0.8 (MI=0.8 group). Immediately after intravenous (IV) injection of Evans Blue®, a single IV dose of PS101 (5.1 mg PFMCP/kg [1000 μL PS101/kg])) was administered, followed by 45 second activation ultrasound (at 2.5 MHz, MI 0.4) and subsequently 5 minute enhancement ultrasound (at 0.5 MHz, MI 0.8), focused to the tumour.

For the MI=0.8 group, tumours and non-insonated control muscle from the right leg were excised and the amount of Evans Blue® was measured by pectrophotometry at 620 nm. The amount of Evans Blue® that was detected in the tumours of the animals in the MI=0.8 group was lower than the amount that was detected in other groups with lower enhancement ultrasound MI. This indicates either potential damage to the cells in the tumour, or damage to the vasculature which reduces the blood supply and, as a result, the concentration of Evans blue in the target volume. It thus appears that when subjected to an enhancement MI of 0.8, the ACT bubbles are causing tissue ablation and significant haemorrhaging. For the MI=0.8 group, all animals died during or immediately after treatment and the insonated area (left thigh and leg) showed extensive haemorrhage. These animals likely died due to high power ultrasound insonation of a significant portion of the mouse body weight (approximately 10%, around 20 times a typical clinical situation) in combination with a high dose of PS101(20 to 40 times the anticipated clinical dose). It is expected that the ablation can be achieved without causing these effects by optimizing the dose of PS101 and the MI of the enhancement ultrasound.

Having described preferred examples of the invention it will be apparent to those skilled in the art that other embodiments incorporating the invention may be used. These and other examples of the invention illustrated above are intended by way of example only and the actual scope of the invention is to be determined from the appended claims.

1-18. (canceled)

19. A method of enhanced ultrasound ablation comprising:
creating at least one ablation-assisting bubble proximate to a target region by:
administering a cluster composition, comprising a microbubble component and a microdroplet component, to a subject, wherein the cluster composition comprises at least one cluster; and
activating a phase shift transition of the microdroplet component of the at least one cluster by ultrasound insonation to create the at least one ablation-assisting bubble;
wherein an expansion from the transition of the at least one cluster to the at least one ablation-assisting bubble provides a mechanical stress on the target region to aid ablation on target tissue in the target region.

20. The method of claim 19, wherein the at least one ablation-assisting bubble has a diameter of at least 10 micrometers.

21. The method of claim 19, further comprising insonating the at least one ablation-assisting bubble with ultrasound at a predetermined intensity to induce at least one of energy absorption, deposition, oscillation and cavitation of the ablation-assisting bubble to provide an additional mechanical and/or thermal stress on the target region, to aid ablation on the target tissue in the target region.

22. The method of claim 21, wherein the predetermined intensity of the ultrasound for insonating the ablation-assisting bubble is equal to an intensity for non-bubble assisted ultrasound ablation divided by a factor of between 12 and 24.

23. The method of claim 19, wherein the enhanced ultrasound ablation is configured to provide a resultant temperature in the target region of 30 to 70 degrees Celsius, for a continuous exposure time of at least 30 seconds.

24. The method of claim 19, further comprising using the at least one ablation-assisting bubble for real-time imaging of the target region by insonating the at least one ablation-assisting bubble with imaging ultrasound and wherein the at least one ablation-assisting bubble is induced as a hyperechoic spot.

25. The method of claim 19, further comprising a further step of ultrasound planning comprising using unique subject and application specific information to plan a specific ultrasound exposure regime wherein ultrasound planning comprises at least one of a passive ultrasound planning and an active ultrasound planning.

26. The method of claim 25, wherein passive ultrasound planning is based on one or more of: physiological information, anatomical structures in an ablation zone, cross-modality imaging and co-registration, and data defining subject anatomy retrieved from a software.

27. The method of claim 25, wherein active ultrasound planning comprises:
monitoring the ablation zone during the enhancement step for real-time feedback of the ablation zone; and
adjusting the specific ultrasound exposure regime to achieve predetermined parameters in the ablation zone;
wherein the steps of monitoring and adjusting are performed on a continuous basis for a predetermined duration.

28. The method of claim 27, wherein monitoring the ablation zone during the enhancement step for real-time feedback of the ablation zone comprises at least one of: real-time temperature feedback, real-time mechanical feedback, cross-modality imaging and co-registration, real-time monitoring of cluster dynamics and concentration, and real-time monitoring of ablation-assisting bubble dynamics and concentration.

29. The method of claim 27, wherein monitoring the target region, including an ablation zone, further comprises imaging via image guidance modalities, wherein the image guidance modalities comprise at least one of: magnetic resonance guided, ultrasound guided, computer tomography guided, optical guided, thermocouple guided, and contrast-enhanced ultrasound guided.

30. The method of claim 19, further comprising co-administration of a therapeutic agent configured to assist ablation efficacy, the therapeutic agent pre-, and/or co- and/or post administered separately to the cluster composition.
